(19) United States
(12) Patent Application Publication
(10) Pub. No.: US 2012/0184748 A1
(43) Pub. Date: Jul. 19, 2012

(54) RECYCLABLE CATALYSTS FOR ESTERIFICATION OR ACYLATION OF ALCOHOLS

(75) Inventors: NORMAN LU, NEW TAIPEI CITY (TW); WEN-HAN TU, NEW TAIPEI CITY (TW); CHIEH-KENG LI, NEW TAIPEI CITY (TW)

(73) Assignee: NATIONAL TAIPEI UNIVERSITY OF TECHNOLOGY, TAIPEI (TW)

(21) Appl. No.: 13/115,245

(22) Filed: May 25, 2011

(30) Foreign Application Priority Data
Jan. 18, 2011 (TW) 100101811

(51) Int. Cl. C07C 67/08; C07D 417/02

(52) U.S. Cl. 546/271.1; 560/231; 560/254; 560/129; 560/249

(57) ABSTRACT

A compound is useful as a recyclable catalyst for esterification or acylation of alcohols and consists of saccharine and a compound comprising a pyridine moiety. In addition, also a method of preparing the compound and an ester synthesis method using the compound are introduced.

### CROSS-REFERENCE TO RELATED APPLICATION

This non-provisional application claims priority under 35 U.S.C. §119(a) on Patent Application No(s). 100101811 filed in Taiwan, R.O.C. on Jan. 18, 2011, the entire contents of which are hereby incorporated by reference.

### FIELD OF TECHNOLOGY

The present invention is related to a recyclable catalyst, especially to a recyclable catalyst for esterification or acylation of an alcohol.

### BACKGROUND

From a viewpoint of atomic economy, an ester is ideally prepared by catalytic condensation of an alcohol with equal equivalent of carboxylic acid. However, such method may encounter some difficulties in case that the alcohol is a tertiary alcohol with higher steric hindrance, or an alcohol with poor nucleophilicity (such as phenol, ally alcohol, amino alcohol, etc.). Accordingly, other efficient methods for synthesis of an ester are still strongly desired in this art.

4-(N,N-dimethylamino)-pyridine(DMAP) is an effective nucleophilic base catalyst which can catalyze esterification of an alcohol with an acid anhydride and lots of other reactions. As the acid anhydride has higher reactivity than that of a carboxylic acid, it is possible for the acid anhydride to acylate a less reactive alcohol to form an ester under catalysis of DMAP. However, DMAP has strong dermatologic toxicity; therefore, it should be used carefully and is preferably recovered after use.

For DMAP recovery, several methods were proposed in prior art. For example, a thesis under the title of “A Magnetic-Nanoparticle-Supported 4-N,N-Dialkylamino pyridine Catalyst: Excellent Reactivity Combined with Facile Catalyst Recovery and Recyclability” published in Angew. Chem. Int. Ed. 2007, 46, 4329-4332 proposed a method for recovering a catalyst composed of a DMAP derivative, comprising binding a DMAP derivative to a carrier composed of magnetic nanoparticles to form the catalyst, and after completion of the reaction, magnetically recovering the catalyst for reuse. However, this method is inconvenient since the DMAP derivative should be bound to the magnetic nanoparticles by a complicated procedure before use.

In another aspect, a thesis under the title of “Fluorous 4-N,N-Dimethyl aminopyridine (DMAP) Salts as Simple Recyclable Acylation Catalysts” published in Chem. Eur. J. 2010, 16, 1776-1779 proposed a catalyst comprising a salt of DMAP with a fluorine-containing acid, which can catalyze esterification of an alcohol with an acid anhydride and can be recovered by precipitation for reuse after completion of the reaction. Nevertheless, such catalyst is not suitable for use in large-scale industrial production since the fluorine-containing acid has high production cost.

In view of the above, a catalyst for esterification of an alcohol with an acid anhydride, which is preferably convenient in use and recyclable, is desired to overcome the disadvantages of prior art.

### SUMMARY

In view of the disadvantages of prior art, one of the main objects of the present invention is to provide a catalyst for catalyzing esterification or acylation of an alcohol, which has advantages of easy recovery and low cost.

It is another object of the present invention to provide a compound consisting of saccharine and a compound comprising a pyridine moiety. The compound can be used as a catalyst, especially as a catalyst for esterification or acylation of an alcohol.

It is further another object of the present invention to provide an ionic compound consisting of saccharine and a compound comprising a pyridine moiety, wherein the compound comprising a pyridine moiety is selected from a group consisting of the following compounds:

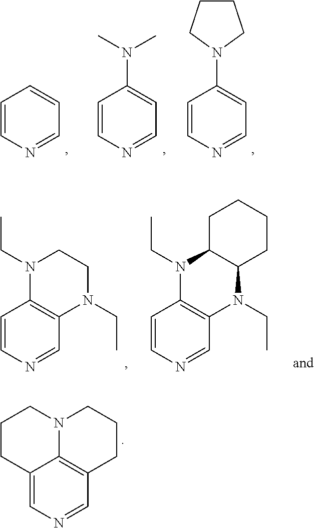

It is still a further object of the present invention to provide a recyclable catalyst for esterification or acylation of an alcohol, comprising an anion derived from an unsubstituted saccharine and a cation derived from a substituted or unsubstituted pyridine in its chemical structure.

It is also an object of the present invention to provide a method for synthesis of an ester, comprising: charging an alcohol and an acid anhydride to a reaction vessel, and adding a salt catalyst to the reaction vessel to catalyze esterification of the alcohol; wherein the salt catalyst comprising an anion derived from saccharine and a cation derived from a compound comprising a pyridine moiety. The method may further comprise precipitating the salt catalyst by using a non-polar solvent, a C₅˜C₁₂ alkane or toluene.

It is also another object of the present invention to provide a method for preparing a catalyst consisting of saccharine and a compound comprising a pyridine moiety. The method comprises: dissolving the saccharine and the compound comprising a pyridine moiety in a solvent; and heating the resulting solution to allow the saccharine and the compound comprising a pyridine moiety to form a salt. The method may further comprise a step of crystallizing the salt.

Other objects, advantages and novel features of the present invention are apparent from the following detailed description and Examples.

### DETAILED DESCRIPTION

In order to elucidate the objects, features and effects of the present invention such that a person having ordinary knowledge in the art that the present invention pertains to, can understand the technical content of the present invention and accordingly practice the present invention; herein below, the present invention is full described with reference to the following embodiments and the attached drawings.

In one embodiment of the present invention, provided is an ionic compound consisting of saccharin (as an anion) and a compound comprising a pyridine moiety (as an cation). In a preferred embodiment, the saccharine may be substituted or unsubstituted, and the compound comprising a pyridine moiety is preferably selected from a group consisting of the following compounds:

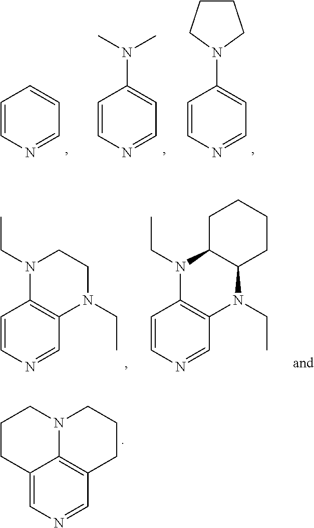

The compound according to the present invention is preferably used as a recoverable catalyst, more preferably used as a recoverable salt catalyst for esterification or acylation of an alcohol. In addition, the compound comprising a pyridine moiety is preferably selected from a group consisting of 4-(pyrrolidin-1-yl)pyridine and 4-(N,N-dimethylamino)-pyridine; more preferably, is 4-(N,N-dimethylamino)-pyridine.

In synthesis of an ester, any one of the compounds of the above embodiments can be added as a catalyst to a reaction vessel with an alcohol and an acid anhydride contained therein, thereby catalyzing the synthesis of the ester. After completion of the reaction, the catalyst can be recovered by precipitating the catalyst through changing its solubility, and then decanting the supernatant (non-solid) to collect the catalyst (solid) for recycle use.

In general, the solubility of the catalyst can be changed in various ways. In a preferred embodiment of the present invention, the catalyst is precipitated by using a non-polar solvent, for example, a C₅-C₁₂ alkane such as pentane, hexane, heptane, octane, etc. In another preferred embodiment, the catalyst is precipitated by using toluene.

In an embodiment of the present invention, when a catalyst consisting of an anion derived from saccharine and a cation derived from a compound comprising a pyridine moiety is used in catalyzing esterification or acylation, good yield can be obtained in absence of a solvent and a base; in other words, the esterification or acylation can be carried out under a neat or solvent-free and base-free condition.

In an embodiment of the present invention; the catalyst consisting of an anion derived from saccharine and a cation derived from a compound comprising a pyridine moiety is synthesized in two steps. First, about equal equivalents of saccharine and the compound comprising a pyridine moiety are dissolved in a solvent (for example, tetrahydrofuran(THF), but not limited thereto); next, the resulting solution is heated to allow the saccharine and the compound comprising a pyridine moiety to form a salt. After completion of the reaction, the reaction mixture is stirred overnight at about 60° C. and concentrated to dryness to obtain the product as white solid. Furthermore, the product may be further purified by crystallization. For example, the product is dissolved in a solvent (for example, methanol, but not limited thereto), then a non-polar solvent, a C₅-C₁₂ alkane or toluene is overlaid on the above solvent to allow diffusive crystallization of the product.

Herein below, the examples according to the present invention and the examples for comparison are provided.

### Example 1

### Preparation of the Catalyst

DMAP (4.09 mmol, 0.5 g) and equal equivalent of saccharine (4.09 mmol, 1.25 g) were charged into a 100 mL of round-bottom flask. 20 ml of THF (as solvent) was then added to the flask to dissolve the above reactants. The resulting reaction mixture was stirred overnight at 60° C. After removing the solvent, a crude product (4.06 mmol, 1.24 g) as white solid was obtained with a yield of 99%. The crude product was purified by dissolving the crude product in methanol in such a way that a saturated solution of the crude product was formed, then overlaying a layer of hexane over the saturated solution to allow the crude product to perform diffusive crystallization. The analytical data of the catalyst thus obtained was as follows:

yield after purification: 95%

melting point: 218° C.

¹H-NMR (500 MHz, D₂O), δ(ppm) H of pyridine ring: 7.90 (d, H₂, ³J_{HH}=7.5 Hz, 2H), 6.72 (d, H₃, ³J_{HH}=7.59 Hz, 2H), 3.12 (s, CH₃, 6H), H of saccharine: 7.76-7.71 (multiplet, 4H)

¹³C-NMR (126 MHz, D₂O), δ(ppm) 39.6, 107.0, 142.3, 157.6 (DMAP C), 120.6, 123.9, 132.7, 133.6, 134.1, 138.3, 172.6 (C of saccharine)

FT-IR v (cm⁻¹) 3077s (N—H), 1646s (C═O, stretching), 1542, 1443m (pyridine), 1329, 1163, 1130, (R—SO₂—N), 1270s (N—CH₃) element analysis (C₁₄H₁₅N₃O₃S): calculated: C, 55.07; H, 4.95; N, 13.76. founded: C, 54.50; H, 4.849; N, 13.50.

### Example 2

### Esterification by Using the Catalyst of Example 1

An alcohol (2 mmol) and an acid anhydride (2.2 mmol) were mixed in a 10 mL of test tube and 1 mol % of the catalyst (0.02 mmol) of Example 1 was added. Then, the test tube was connected to a vacuum manifold (Schlenk line (or with a cover) and the reaction mixture was stirred at room temperature (or at 60° C. if the alcohol is 1-methylcyclopentanol). After several hours (2 to 12 hours, depending on the reactants used) of reaction time, the acid formed was vaporized off in vacuum. The residue was cooled to room temperature and 2 ml of hexane or toluene was added to precipitate the catalyst. The catalyst was recovered by filtration, and the filtrate was vaporized to remove the solvent, thereby obtaining the ester product. The recovered catalyst can be reused in catalyzing the reaction of the subsequent run.

### Example 3

### Esterification of Secondary Alcohols by Using the Method of Example 2

Various secondary alcohols were esterified according to the following scheme by using the method of Example 2, and the results were listed in Table 1.

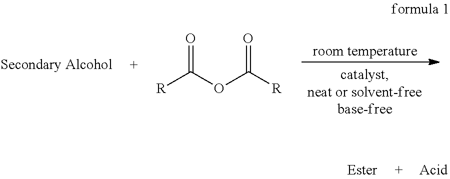

TABLE 1

| | | | Temp. | Time | |
|---|---|---|---|---|---|
| No. | Sec. Alcohol | R | (° C.) | (hr.) | Yield (%) |
| 1 | 1-cylco- | Me | 25 | 2.5 | 99.5, 99.1, 99.7, 96.6, |
| | hexanol | | | | 97.4, 96.8, 97.6, 93.5, |
| | | | | | 92.6, 86.6 |
| 2 | 1-cylco- | iPr | 25 | 2 | >99.5, >99.5, >99.5, >99.5, |
| | hexanol | | | | >99.5, >99.5, 99, 99, 98, |
| | | | | | 98 |
| 3 | 1-phenyl- | Me | 25 | 2 | >99.5, >99.5, >99.5, >99.5, |
| | ethanol | | | | >99.5, >99.5, >99.5, >99.5, |
| | | | | | >99.5, >99.5 |
| 4 | 1-phenyl- | iPr | 25 | 2 | >99.5, >99.5, >99.5, >99.5, |
| | ethanol | | | | >99.5, >99.5, >99.5, >99.5, |
| | | | | | >99.5, >99.5 |
| 5 | menthol | Me | 25 | 8-12 | 97, 96, 96, 96, 95, 92, 95, |
| | | | | | 98, 92, 87 |
| 6 | menthol | iPr | 25 | 8-12 | 98, 98, 96, 96, 97, 95, 97, |
| | | | | | 99, 95, 92 |
| 7 | 1-cyclo- | Me | 25 | 8 | 99, 98, 99, 97, 97, 97, 96, |
| | dodecanol | | | | 97, 97 |
| 8 | 1-cyclo- | iPr | 25 | 8 | 95, 95, 95, 95, 94, 94, 95, |
| | dodecanol | | | | 95 |
| 9 | 4-nitro-phenol | Me | 25 | 2-4 | >99.5, >99.5, >99.5, >97, |
| | | | | | >99.5, >99.5, 97.5, 96.4, |
| | | | | | 96, 96 |
| 10 | 4-nitro-phenol | iPr | 25 | 2-4 | 99.5, 99.5, 99.5, 99.5, |
| | | | | | 99.5, 99.5, 99.5, 99.5, |
| | | | | | 99.5, 99.5 |

From the results of No. 1 and No. 2 in Table 1, it can be known that the catalyst of Example 1 can catalyze the esterification of secondary alcohols. Since it is more difficult for a secondary alcohol to be esterified than a primary alcohol, the catalyst of Example 1 can surely catalyze the esterification of a primary alcohol. In addition, the catalyst of Example 1 can catalyze the reaction of an alcohol not only with acetic anhydride (see No. 1), but also with isobutyric anhydride, which has higher steric hindrance (see No. 2). Furthermore, it can be known from the yields that the effect of the catalyst did not decrease even after the catalyst was reused many times. For example, as shown by the results of No. 2 in Table 1, the yield was still as high as 98% after the catalyst was reused 10 times.

From the results of No. 3 to 10, it can be known that the catalyst of Example 1 can also catalyze the reaction of secondary alcohols other than 1-cyclohexanol with various acid anhydrides. 1-phenylethanol (Nos. 3 and 4) can be esterified with high yield under substantially the same conditions as those for esterification of 1-cyclohexanol. For menthol (Nos. 5 and 6), longer reaction time (about 8 hours) was needed since menthol had higher steric hindrance than that of 1-cyclohexanol. For 1-cyclododecanol (Nos. 7 and 8), the reaction time is about 8 hours, and high yield was obtained even after the catalyst had been reused 8 times. In addition, for phenols with lower nucleophilicity, such as 4-nitrophenol (Nos. 9 and 10), the catalyst also showed good catalytic effect on acylation thereof since the reaction can be completed in 4 hours and the average yield was still above 98% even after the catalyst was reused 10 times.

### Comparative Example 1

### Esterification of Secondary Alcohols by Using Other Methods

The results of esterification of secondary alcohols by using other catalytic method were shown in Table 2, wherein No. 1 to 3 is performed according to the following literatures and the catalysts were used in an amount of 10 mol %, 7.5 mol % and 5 mol %, respectively.

No. 1: D. Vuluga, J. Legros, B. Crousse, D. Bonnet-Delpon, Chem. Eur. J. 2010, 16, 1776.

No. 2: a) C. Ó. Dálaigh, S. A. Corr, Y. Gun'ko, S. J. Connon, Angew. Chem. 2007, 119, 4407; b) Angew. Chem. Int. Ed. 2007, 46, 4329.

No. 3: H.-T. Chen, S. Huh, J. W. Wiench, M. Pruski, and V. S.-Y. Lin, J. Am. Chen. Soc. 2005, 127, 13305-13311.

TABLE 2

| | | | Temp. | Time | |
|---|---|---|---|---|---|
| No. | Sec. Alcohol | R | (° C.) | (hr.) | Yield (%) |
| 1 | 1-cylco-hexanol | Me | 25 | 8 | 85 |
| 2 | 1-cylco-hexanol | Me | 60 | 2.5 | 90 |
| 3 | 1-phenyl-ethanol | Me | 25 | 16 | >98, 94, 97, >98, >98, |
| | | | | | >98, >98, 97, 98, 97 |

Comparing the results of No. 1 listed in Table 2 with the results of No. 1 listed in Table 1, it can be known that the method of Legros et al. using DMAP-R_{f}COOH as catalyst, not only needed higher amount (10 mol %) of the catalyst and more reaction time (8 hours), but also had lower yield (85%). Since the method of Example 1 according to the present invention only needed ⅓ of the reaction time and 1/10 of the catalyst amount when compared with the method of Legros et al., the catalyst of Example 1 according to the present invention has a catalytic efficiency at least 30 times higher than that of DMAP-R_{f}COOH. In addition, it can be known that from the results of Nos. 2 and 3 listed in Table 2, the catalyst of Example 1 according to the present invention is also superior to the heterophasic catalytic system using nanoparticles.

### Comparative Example 2

### Esterification of an Alcohol by Using Saccharine as Catalyst or in Absence of an Catalyst

As shown in Table 1, the esterification of 1-cyclohexanol with acetic anhydride can be completed in about 2 hours when the catalyst of Example 1 according to the present invention was used. In contrast, esterification was not significant with a conversion rate as low as 10% after 3 hours when saccharine (a weak acid) was used as catalyst. In addition, esterification did not occur in absence of a catalyst.

### Example 4

### Esterification of Tertiary Alcohols by Using the Method of Example 2

2-phenyl-2-propanol, a tertiary alcohol, was esterified according to the method of above Example 2, and the results were shown in Table 3.

TABLE 3

| | | | Temp. | Time | |
|---|---|---|---|---|---|
| No. | Tert. Alcohol | R | (° C.) | (hr.) | Yield (%) |
| 1 | 2-phenyl-2- | Me | 100 | 24 | 90, 90, 88, 85, 88, 88, |
| | propanol | | | | 88, 88, 85, 80 |
| 2 | 2-phenyl-2- | iPr | 100 | 24 | >99.5, >99.5, >99.5, 99, |
| | propanol | | | | 96, 94, 93, 91, 90, 89 |

It can be known from Table 3 that tertiary alcohols, which usually have higher steric hindrance, can be esterified by using the catalyst of Example 1 if the reaction temperature is elevated and the reaction time is prolonged.

### Example 5

### Recovery of the Catalyst

This example is used to illustrate the high recovery rate of the catalyst of Example 1. 1-Cyclohexanol (50 mmol, 200.3 mg) was esterified with acetic anhydride in the presence of the catalyst (1 mol %, 152.7 mg) prepared according to the method of Example 1 (in absence of any solvent, at a temperature of 25° C.). After completion of esterification, the catalyst was recovered and weighed. The above procedures were repeated 8 times and the results were shown in Table 4. It can be known from the data reported in Table 4 that the recovery rate of the catalyst was above 98% each time.

TABLE 4

| No. of | Amount of Recovered | Recovery | |
|---|---|---|---|
| Reaction | Catalyst (g) | Rate (%) | Yield (%) |
| 1 | 0.151 | 99.3 | >99 |
| 2 | 0.150 | 99.3 | >99 |
| 3 | 0.148 | 98.7 | >99 |
| 4 | 0.146 | 98.6 | >99 |
| 5 | 0.144 | 98.6 | >99 |
| 6 | 0.142 | 98.6 | >99 |
| 7 | 0.140 | 98.6 | >99 |
| 8 | 0.138 | 98.6 | >99 |

The preferred embodiments of the present invention have been described above; however, it should be understood by the persons having ordinary knowledge in this art that these embodiments are merely used to illustrate the present invention but not intended to limit the present invention thereto. It should be noted that various equivalent modifications or replacements made on these embodiments are all embraced in the scope of the present invention. Accordingly, the protection scope of the present invention patent shall be defined by the appended claims.

What is claimed is:

1. A compound consisting of saccharine and a compound comprising a pyridine moiety.

2. The compound according to claim 1, which is used as a catalyst.

3. The compound according to claim 2, which is used as a catalyst for esterification or acylation of an alcohol.

4. The compound according to claim 1, wherein the compound comprising a pyridine moiety is selected from a group consisting of the following compounds:

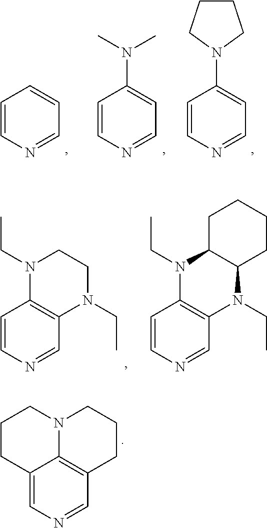

5. The compound according to claim 4, wherein the compound comprising a pyridine moiety is selected from a group consisting of 4-(pyrrolidin-1-yl)pyridine and 4-(N,N-dimethylamino)-pyridine.

6. The compound according to claim 4, which is a salt catalyst.

7. The compound according to claim 6, wherein the compound comprising a pyridine moiety is 4-(N,N-dimethylamino)-pyridine.

8. The compound according to claim 1, wherein the saccharine is unsubstituted.

9. A recyclable catalyst for esterification or acylation of an alcohol, comprising an anion derived from an unsubstituted saccharine and a cation derived from a substituted or unsubstituted pyridine in its chemical structure.

10. The recyclable catalyst according to claim 9, wherein the cation is 4-(N,N-dimethylamino)-pyridine.

11. A method for synthesis of an ester, comprising
charging an alcohol and an acid anhydride to a reaction vessel; and
adding a salt catalyst to the reaction vessel to catalyze esterification of the alcohol;
wherein the salt catalyst comprises an anion derived from saccharine and a cation derived from a compound comprising a pyridine moiety.

12. The method according to claim 11, wherein the compound comprising a pyridine moiety is selected from a group consisting of the following compounds:

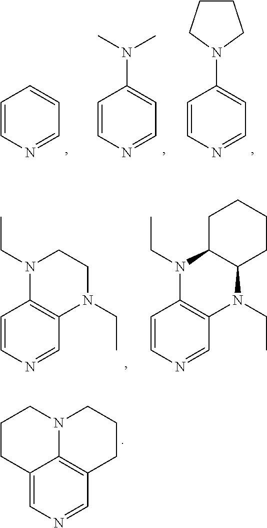

13. The method according to claim 11, wherein the compound comprising a pyridine moiety is selected from a group consisting of 4-(pyrrolidin-1-yl)pyridine and 4-(N,N-dimethylamino)-pyridine.

14. The method according to claim 13, wherein the saccharine is unsubstituted.

15. The method according to claim 11, further comprising a step of precipitating the salt catalyst by using a non-polar solvent.

16. The method according to claim 11, further comprising a step of precipitating the salt catalyst by using a C₅-C₁₂ alkane or toluene.

17. The method according to claim 11, wherein the esterification of the alcohol is performed at a temperature equivalent to or higher than room temperature.

18. The method according to claim 11, wherein the esterification of the alcohol is performed under a base-free and neat or solvent-free condition.

19. A method for preparing a catalyst consisting of saccharine and a compound comprising a pyridine moiety, comprising:
dissolving the saccharine and the compound comprising a pyridine moiety in a solvent; and
heating the resulting solution to allow the saccharine and the compound comprising a pyridine moiety to form a salt.

20. The method according to claim 19, wherein the compound comprising a pyridine moiety is selected from a group consisting of 4-(pyrrolidin-1-yl)pyridine and 4-(N,N-dimethylamino)-pyridine.

21. The method according to claim 19, further comprising a step of crystallizing the salt.
